(19) United States
(12) Patent Application Publication
(10) Pub. No.: US 2012/0122982 A1
(43) Pub. Date: May 17, 2012

(54) USE OF PUFAS TO TREAT NERVE DAMAGE

(75) Inventors: Adam Kelliher, London (GB); Norman Camaron, Aberdeen (GB); Angus Morrison, Isle of Lewis (GB); Phil Knowles, Cumbria (GB)

(73) Assignee: Equateq Limited, London (GB)

(21) Appl. No.: 13/318,420

(22) PCT Filed: Apr. 22, 2010
(86) PCT No.: PCT/GB2010/000817

§ 371 Date: Jan. 24, 2012

(30) Foreign Application Priority Data
May 1, 2009 (GB) 0907601.9

(51) Int. Cl. A61K 31/201; A61P 25/00; A61P 25/02; A61P 13/10; A61P 31/00; A61P 29/00; A61P 15/00; A61P 43/00; A61P 1/00; A61K 31/202; A61P 21/00

(52) U.S. Cl. 514/560

(57) ABSTRACT

The present invention provides use of compounds which are polyunsaturated fatty acid (PUFA) derivatives of formula (I), in the form of racemates, stereoisomers or mixtures of stereoisomers, or pharmaceutically acceptable salts, or solvates thereof, wherein -AIk- is —(CH₂)₄—CH(OR₂)-[trans]CH═CH-[cis]CH═CH—, —(CH₂)₄-[cis]CH═CH-[trans]CH═CH—CH(OR₂)—, —CH(OR₂)-[trans]CH═CH-[cis]CH═CH—CH₂-[cis]CH═CH—(CH₂)₃—, —(CH₂)₃—CH(OR₂)-[trans]CH═CH-[cis]CH═CH—CH₂-[cis]CH═CH—, or —(CH₂)₃-[cis]CH═CH—CH₂-[cis]CH═CH-[trans]CH═CH—CH(OR₂)—; R₁ is a hydrogen atom; or R₁ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group; or R₁ is a group of formula —CH₂—CH(OR₃)—CH₂—(OR₄), wherein R₃ and R₄ are each independently hydrogen atoms or —(C═O)—R₆, wherein R₆ is an aliphatic group having from 3 to 29 carbon atoms; or R₁ is a group of formula —(CH₂OCH₂)ₘOH, wherein m is an integer of from 1 to 200; or R₁ is a drug moiety; each R₂ is the-same or different- and each independently represents a hydrogen atom; or a group —(C═O)—R₅, wherein R₅ is a C₁-C₆ alkyl, C₂-C6 alkenyl, C₂-C₆ alkynyl, C₆-C₁₀ aryl, 5- to 10-membered heteroaryl, C₃-C₇ carbocyclyl or 5- to 10-membered heterocyclyl group, or R₅ is an aliphatic group having from 3 to 29 carbon atoms, or R₅ is a drug moiety; or a group of formula —(CH₂OCH₂)ₙOH, wherein n is an integer of from 1 to 200; or a drug moiety; and wherein said alkyl, alkenyl, alkynyl and aliphatic groups are the same or different and are each unsubstituted or substituted with 1, 2 or 3 unsubstituted substituents which are the same or different and are selected from halogen atoms and C₁-C₄ alkoxy, C₂-C₄ alkenyloxy, C₁-C₄ haloalkyl, C₂-C₄ haloalkenyl, C₁-C₄ haloalkoxy, C₂-C₄ haloalkenyloxy, hydroxyl, —SR′, and —NR′R″ groups where R′ and R″ are the same or different and represent hydrogen or unsubstituted C₁-C₂ alkyl; said aryl, heteroaryl, carbocyclyl and heterocyclyl groups are the same or different and are each unsubstituted or substituted by 1, 2, 3 or 4 unsubstituted substituents which are the same or different and are selected from halogen atoms, and cyano, nitro, C₁-C₄ alkyl, C₁-C₄ alkoxy, C₂-C₄ alkenyl, C₂-C₄ alkenyloxy, C₁-C₄ haloalkyl, C₂-C₄ haloalkenyl, C₁-C₄ haloalkoxy, C₂-C₄ haloalkenyloxy, hydroxyl, C₁-C₄ hydroxyalkyl, —SR′ and —NR′R″ groups wherein each R′ and R″ is the same or different and represents hydrogen or unsubstituted C₁-C₄ alkyl; in the manufacture of a medicament for use in treating or preventing nerve damage in a mammal.

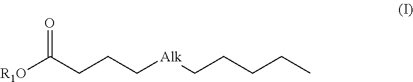

### FIELD OF THE INVENTION

The present invention relates to novel methods of treating and/or preventing nerve damage in mammals, particularly nerve damage in patients suffering from diabetes, i.e. diabetic neuropathy.

### BACKGROUND OF THE INVENTION

Nerve damage in mammals can result from a number of different aetiologies. It may result from, for example, exposure to infectious agents, such as bacteria, viruses or prions, in particular HIV/AIDS; metabolic or mitochondrial disorders, such as diabetes; tumours, in particular brain tumours; genetic diseases; exposure to toxins, for example solvents, drugs, alcohol, paints, industrial chemicals, and certain metals; radiation; chemotherapy; trauma; poor nutrition, for example vitamin deficiency; degenerative conditions, such as Alzheimer's or Parkinson's Disease; inflammatory diseases; or lack of oxygen or blood flow to the nerve cells, for example vaso-occlusive crises caused by sickle cell anaemia.

The mammalian nervous system is divided broadly into two categories: the peripheral nervous system and the central nervous system. The central nervous system comprises the brain and spinal cord. The peripheral nervous system comprises the remainder of the nervous system outside the central nervous system. The peripheral nervous system is further divided into the somatic nervous system and the autonomic nervous system.

Disorders of the peripheral nervous system are commonly referred to as peripheral neuropathy, or simply neuropathy. As mentioned above, there is a wide range of factors known to cause nerve damage in mammals. However, a leading known cause of peripheral neuropathy in humans is diabetes mellitus. Peripheral neuropathy caused by diabetes is commonly referred to as diabetic neuropathy. Diabetic neuropathy is caused by the cumulative effect of irregular blood glucose levels, which disturb and damage the body's nerves.

Patients suffering from diabetic neuropathy typically display negative (loss of function) symptoms and positive (gain of function) symptoms in both their sensory and motor functions. Symptoms include numbness, dysesthesia (decreased or loss of sensation to a body part), dysphagia (difficulty swallowing), speech impairment, tremor, muscle weakness, dizziness, tiredness, heaviness, drooping of the face, mouth or eyelid, vision changes, loss of balance, gait abnormalities, tingling, pain (burning, stabbing, and/or electric shock like pain), itching, crawling sensations, pins and needles, cramps, fasciculations (muscle contractions), and foot sores. Autonomic nerve damage resulting from diabetic neuropathy may result in abnormal blood pressure and heart rate, reduced ability to perspire, gustatory sweating, indigestion, constipation, diarrhea, bladder dysfunction, i.e. incontinence, which can in turn lead to bladder infections, impotence, and sexual dysfunction (e.g. erectile dysfunction). Foot sores are relatively common in patients suffering from diabetic neuropathy and if left untreated, may result in extreme health implications, including limb amputation or mortality. Diabetic neuropathy is the leading cause of morbidity and mortality in diabetic patients.

Current treatments for diabetic neuropathy include tricyclic antidepressant drugs, selective serotonin reuptake inhibitors (SSRIs), anticonvulsant agents and opioid pain-killers. Most available therapies for diabetic neuropathy, however, provide only temporary relief from the distressing symptoms of the condition. Thus, it is not currently possible to target the underlying physical mechanisms of the condition, slow its progression, or regenerate damaged nerves. In addition, many of the available therapies are associated with undesirable side-effects.

Accordingly, there is a need for new methods for treating or preventing nerve damage, in particular for treating or preventing diabetic neuropathy, in mammals. In addition, there is a need for methods which target the nerve damage itself and which slow its progression and aid regeneration of nerves, rather than merely alleviating the symptoms associated with nerve damage.

9-Hydroxyoctadeca-10E,12Z-dienoic acid (9-HODE) is a commercially available polyunsaturated fatty acid (PUFA) derivative derived from octadeca-9E,12E-dienoic acid (Linoleic acid or LA). 9-HODE has the structure shown below.

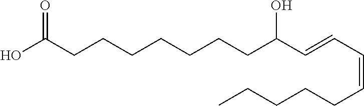

13-Hydroxyoctadeca-9Z,11E-dienoic acid (13-HODE) is a commercially available polyunsaturated fatty acid (PUFA) derivative derived from octadeca-9E,12E-dienoic acid (Linoleic acid or LA). 13-HODE has the structure shown below.

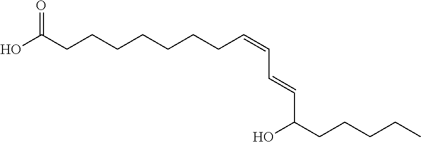

5-Hydroxy-eicosa-6E,8Z,11Z-trienoic acid (5-HETrE)) is a commercially available PUFA derivative derived from mead acid. 5-HETrE has the structure shown below.

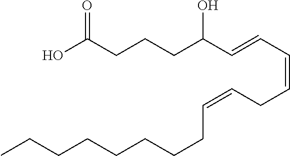

8-Hydroxy-eicosa-9E,11Z,14Z-trienoic acid (8-HETrE) is a commercially available PUFA derivative derived from eicosa-8Z,11Z,14Z-trienoic acid (Dihomo-γ-linolenic acid or DGLA). 8-HETrE has the structure shown below.

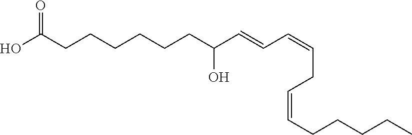

15-Hydroxy-eicosa-8Z,11Z,13E-trienoic acid (15-HETrE) is a commercially available PUFA derivative derived from eicosa-8Z,11Z,14Z-trienoic acid (Dihomo-γ-linolenic acid or DGLA). 15-HETrE has the structure shown below.

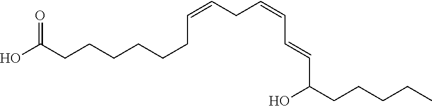

WO-A-0176568 describes 13-HODE as an antithrombotic agent. It does not describe use of 13-HODE in treating or preventing nerve damage in mammals.

It is known to use gamma-linolenic acid (GLA) and other related PUFAs to treat diabetic neuropathy. It has, however, been surprisingly found that the compounds used in the present invention are much more potent in restoring nerve function than GLA. Thus, 13-HODE is approximately 3000 times more potent than GLA in restoring motor nerve conduction velocity in rats. 15-HETrE is approximately 500 times more potent than GLA. Advantageously, this means that the compounds used in the present invention can be administered at a much lower dosage than GLA and other related PUFAs.

It has now been surprisingly found that 9-HODE, 13-HODE, 5-HETrE, 8-HETrE and 15-HETrE and their derivatives are capable of treating or preventing nerve damage, in particular nerve damage associated with diabetic neuropathy.

### SUMMARY OF THE INVENTION

The present invention therefore provides use of compounds which are polyunsaturated fatty acid (PUFA) derivatives of formula (I),

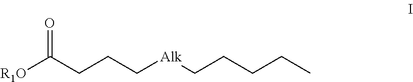

in the form of racemates, stereoisomers or mixtures of stereoisomers, or pharmaceutically acceptable salts or solvates thereof, wherein
-Alk- is —(CH₂)₄—CH(OR₂)-[trans]CH═CH-[cis]CH═CH—, —(CH₂)₄-[cis]CH═CH-[trans]CH═CH—CH(OR₂)—, —CH(OR₂)-[trans]CH═CH-[cis]CH═CH—CH₂-[cis]CH═CH—(CH₂)₃—, —(CH₂)₃—CH(OR₂)-[trans]CH═CH-[cis]CH═CH—CH₂-[cis]CH═CH—, or —(CH₂)₃-[cis]CH═CH—CH₂-[cis]CH═CH-[trans]CH═CH—CH(OR₂)—;
R₁ is a hydrogen atom; or
   R₁ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group; or
   R₁ is a group of formula —CH₂—CH(OR₃)—CH₂—(OR₄), wherein R₃ and R₄ are each independently hydrogen atoms or —(C═O)—R₆, wherein R₆ is an aliphatic group having from 3 to 29 carbon atoms; or
   R₁ is a group of formula —(CH₂OCH₂)ₘOH, wherein m is an integer of from 1 to 200; or
   R₁ is a drug moiety;
each R₂ is the same or different and each independently represents a hydrogen atom; or
   a group —(C═O)—R₅, wherein R₅ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group, or R₅ is an aliphatic group having from 3 to 29 carbon atoms, or R₅ is a drug moiety; or
   a group of formula —(CH₂OCH₂)ₙOH, wherein n is an integer of from 1 to 200; or
   a drug moiety;
      and wherein
said alkyl, alkenyl, alkynyl and aliphatic groups are the same or different and are each unsubstituted or substituted with 1, 2 or 3 unsubstituted substituents which are the same or different and are selected from halogen atoms and C₁-C₄ alkoxy, C₂-C₄ alkenyloxy, C₁-C₄ haloalkyl, C₂-C₄ haloalkenyl, C₁-C₄ haloalkoxy, C₂-C₄ haloalkenyloxy, hydroxyl, —SR′, and —NR′R″ groups where R′ and R″ are the same or different and represent hydrogen or unsubstituted C₁-C₂ alkyl;
said aryl, heteroaryl, carbocyclyl and heterocyclyl groups are the same or different and are each unsubstituted or substituted by 1, 2, 3 or 4 unsubstituted substituents which are the same or different and are selected from halogen atoms, and cyano, nitro, C₁-C₄ alkyl, C₁-C₄ alkoxy, C₂-C₄ alkenyl, C₂-C₄ alkenyloxy, C₁-C₄haloalkyl, C₂-C₄ haloalkenyl, C₁-C₄ haloalkoxy, C₂-C₄ haloalkenyloxy, hydroxyl, C₁-C₄ hydroxyalkyl, —SR′ and —NR′R″ groups wherein each R′ and R″ is the same or different and represents hydrogen or unsubstituted C₁-C₄ alkyl;
   in the manufacture of a medicament for use in treating or preventing nerve damage in a mammal.

Also provided is use of compounds which are polyunsaturated fatty acid (PUFA) derivatives of formula (I),

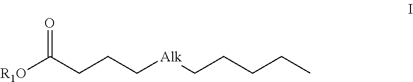

in the form of racemates, stereoisomers or mixtures of stereoisomers, or pharmaceutically acceptable salts, or solvates thereof,
wherein
-Alk- is —(CH₂)₄—CH(OR₂)-[trans]CH═CH-[cis]CH═CH—, —(CH₂)₄-[cis]CH═CH-[trans]CH═CH—CH(OR₂)—, —CH(OR₂)-[trans]CH═CH-[cis]CH═CH—CH₂-[cis]CH═CH—(CH₂)₃—, —(CH₂)₃—CH(OR₂)-[trans]CH═CH-[cis]CH═CH—CH₂-[cis]CH═CH—, or —(CH₂)₃-[cis]CH═CH—CH₂-[cis]CH═CH-[trans]CH═CH—CH(OR₂)—;
R₁ is a hydrogen atom; or
   R₁ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group; or
   R₁ is a group of formula —CH₂—CH(OR₃)—CH₂—(OR₄), wherein R₃ and R₄ are each independently hydrogen atoms or —(C═O)—R₆, wherein R₆ is an aliphatic group having from 3 to 29 carbon atoms; or
   R₁ is a group of formula —(CH₂OCH₂)ₘOH, wherein m is an integer of from 1 to 200; or
   R₁ is a drug moiety;
each R₂ is the same or different and each independently represents a hydrogen atom; or
   a group —(C═O)—R₅, wherein R₅ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group, or R₅ is an aliphatic group having from 3 to 29 carbon atoms, or R₅ is a drug moiety; or
   a group of formula —(CH₂OCH₂)ₙOH, wherein n is an integer of from 1 to 200; or
   a drug moiety;
      and wherein
said alkyl, alkenyl, alkynyl and aliphatic groups are the same or different and are each unsubstituted or substituted with 1, 2 or 3 unsubstituted substituents which are the same or different and are selected from halogen atoms and C₁-C₄ alkoxy, C₂-C₄ alkenyloxy, C₁-C₄ haloalkyl, C₂-C₄ haloalkenyl, C₁-C₄ haloalkoxy, C₂-C₄ haloalkenyloxy, hydroxyl, —SR′, and —NR′R″ groups where R′ and R″ are the same or different and represent hydrogen or unsubstituted C₁-C₂ alkyl;
said aryl, heteroaryl, carbocyclyl and heterocyclyl groups are the same or different and are each unsubstituted or substituted by 1, 2, 3 or 4 unsubstituted substituents which are the same or different and are selected from halogen atoms, and cyano, nitro, C₁-C₄ alkyl, C₁-C₄ alkoxy, C₂-C₄ alkenyl, C₂-C₄ alkenyloxy, C₁-C₄ haloalkyl, C₂-C₄ haloalkenyl, C₁-C₄ haloalkoxy, C₂-C₄ haloalkenyloxy, hydroxyl, C₁-C₄ hydroxyalkyl, —SR′ and —NR′R″ groups wherein each R′ and R″ is the same or different and represents hydrogen or unsubstituted C₁-C₄ alkyl;
   in the manufacture of a medicament for use in treating or preventing dizziness, indigestion, bladder infections, foot sores, wastage of thigh muscles, sexual dysfunction (e.g. erectile dysfunction), numbness, burning sensations, pain, tingling in the legs and feet, decreased or loss of temperature perception, decreased or loss of ankle reflex and/or decreased or loss of sensitivity to vibrations, arising from diabetic neuropathy.

### DETAILED DESCRIPTION OF THE INVENTION

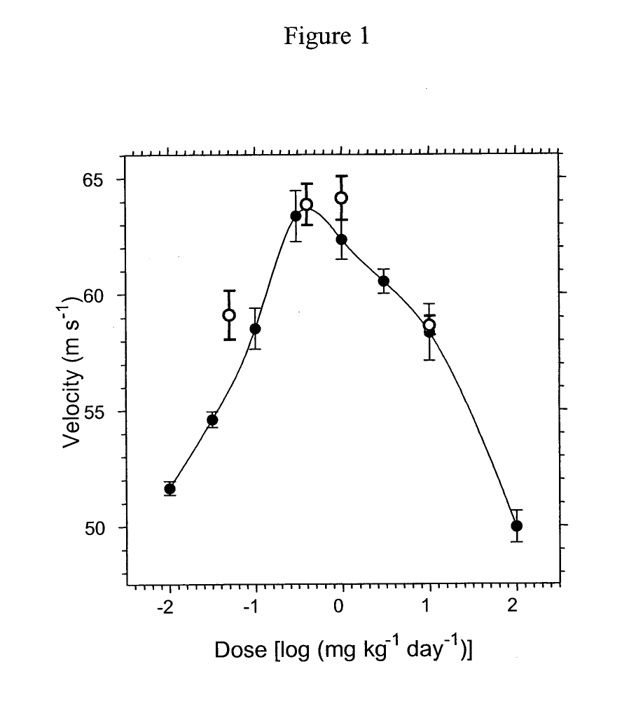

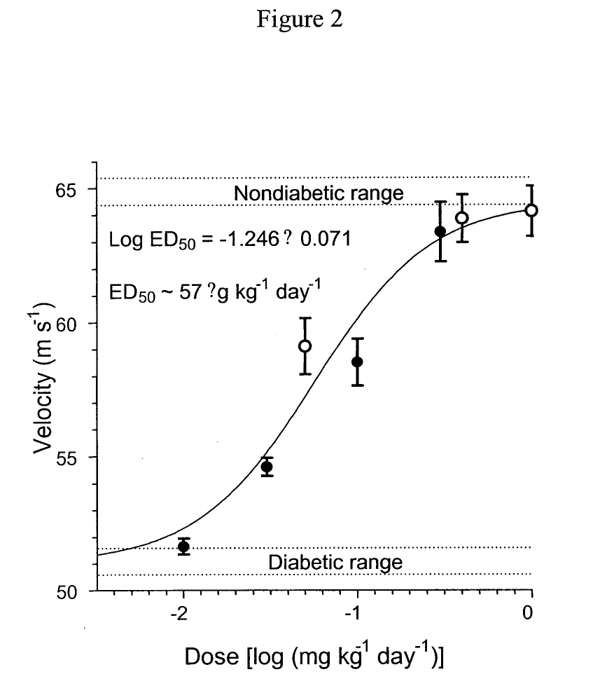

FIGS. 1 and 2 show the results of a nerve conduction velocity (NCV) experiment to determine the effect of daily dosage of 13-HODE on NCV in motor neurons in rats.

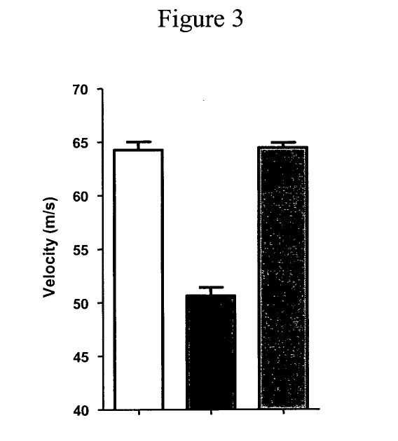

FIG. 3 shows a comparison of motor nerve conduction velocities in non-diabetic rats (first bar), diabetic rats (second bar) and diabetic rats treated with 13-HODE (third bar).

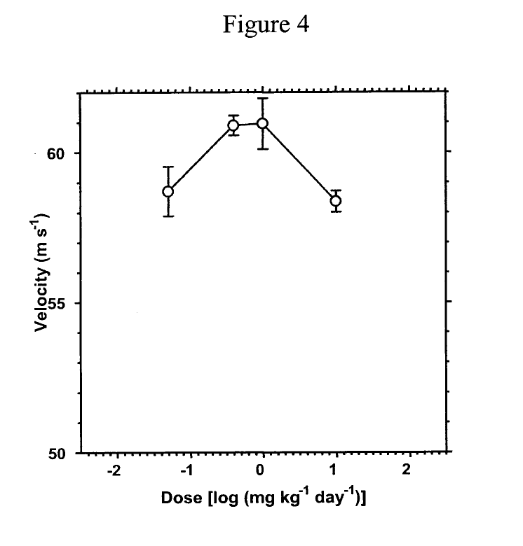

FIG. 4 shows the results of a nerve conduction velocity (NCV) experiment to determine the effect of daily dosage of 13-HODE on NCV in sensory neurons in rats.

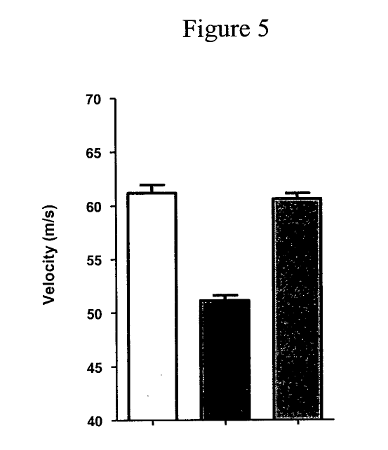

FIG. 5 shows a comparison of sensory nerve conduction velocities in non-diabetic rats (first bar), diabetic rats (second bar) and diabetic rats treated with 13-HODE (third bar).

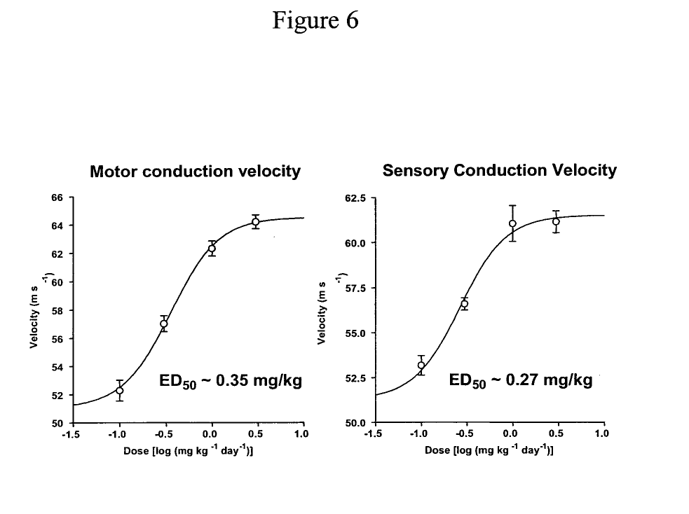

FIG. 6 shows the results of a nerve conduction velocity (NCV) experiment to determine the effect of daily dosage of 15-HETrE on NCV in motor and sensory neurons in rats.

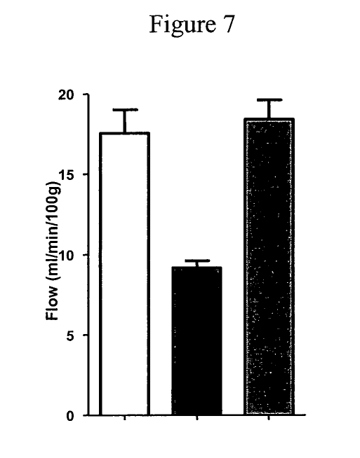

FIG. 7 shows a comparison of sciatic nerve blood flow in non-diabetic rats (first bar), diabetic rats (second bar) and diabetic rats treated with 13-HODE (third bar).

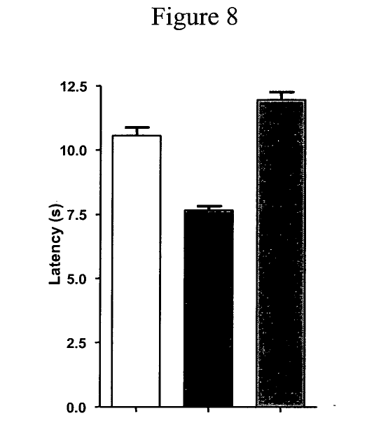

FIG. 8 shows a comparison of latency of response to thermal stimuli in non-diabetic rats (first bar), diabetic rats (second bar) and diabetic rats treated with 13-HODE (third bar).

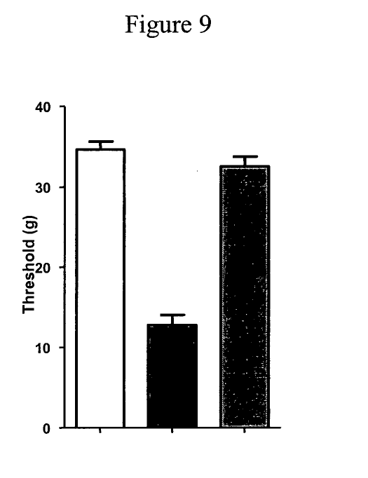

FIG. 9 shows a comparison of tactile allodynia in non-diabetic rats (first bar), diabetic rats (second bar) and diabetic rats treated with 13-HODE (third bar).

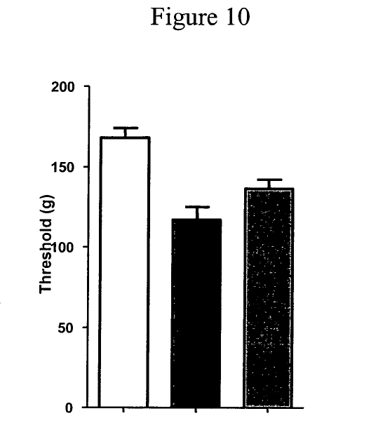

FIG. 10 shows a comparison of foot withdrawal responses to mechanical deep pressure in non-diabetic rats (first bar), diabetic rats (second bar) and diabetic rats treated with 13-HODE (third bar).

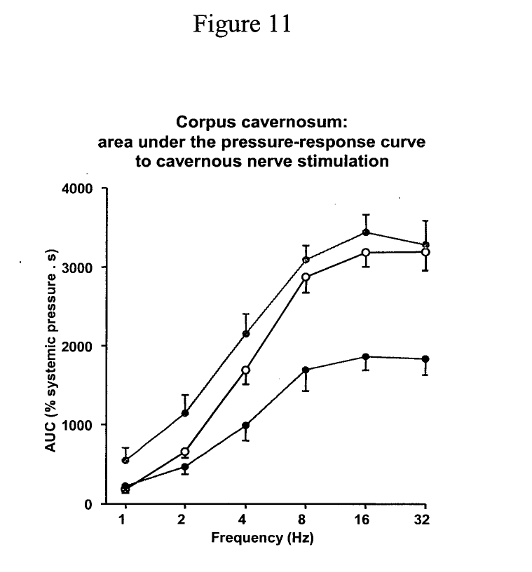

FIG. 11 shows a comparison of corpus cavernosum responses to cavernous nerve stimulation in non-diabetic rats (middle line), diabetic rats (bottom line) and diabetic rats treated with 13-HODE (top line).

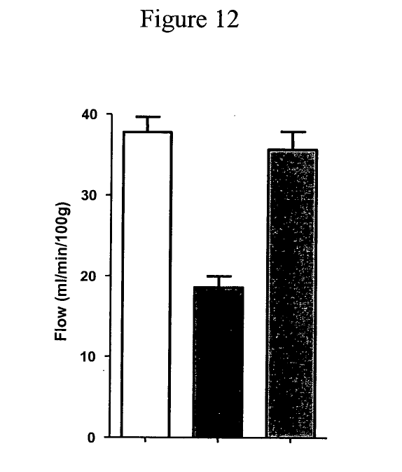

FIG. 12 shows a comparison of major pelvic ganglion blood flow in non-diabetic rats (first bar), diabetic rats (second bar) and diabetic rats treated with 13-HODE (third bar).

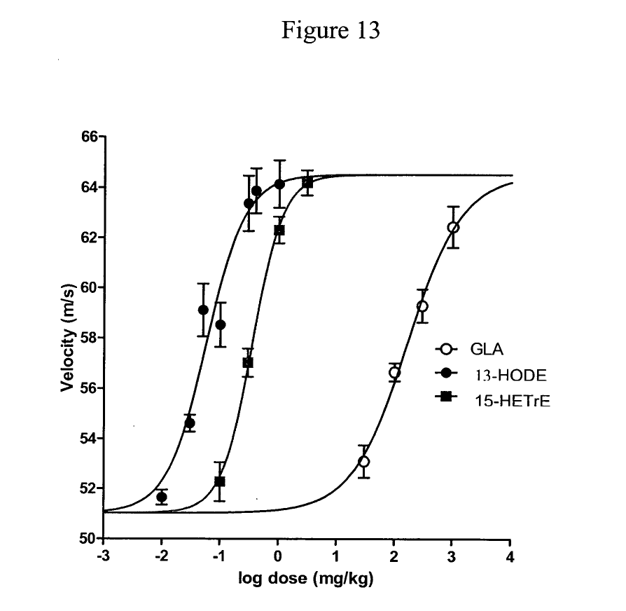

FIG. 13 shows dose response curves of motor NCV in diabetic rats treated with GLA, 13-HODE and 15-HETrE.

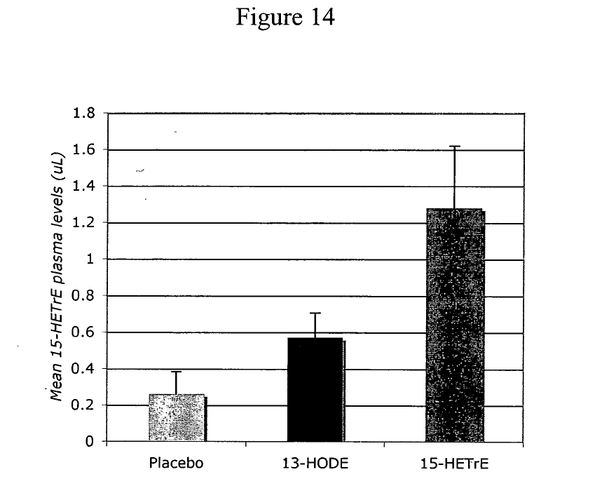

FIG. 14 shows the tissue plasma levels of 15-HETrE in rats treated with (i) 15-HETrE, (ii) 13-HODE and (iii) sunflower oil placebo.

Preferably the alkyl, alkenyl, alkynyl and aliphatic groups are unsubstituted or substituted with 1, 2 or 3, preferably 1 or 2, more preferably 1, unsubstituted substituents which are the same or different and are selected from halogen atoms and C₁-C₄ alkoxy, hydroxyl, C₁-C₄ haloalkyl, C₂-C₄ haloalkenyl, C₁-C₄ haloalkyloxy and —NR′R″ wherein R′ and R″ are the same or different and represent hydrogen or C₁-C₂ alkyl. More preferred substituents are halogen, C₁-C₄ alkoxy, hydroxyl and —NR′R″ groups where R′ and R″ are the same or different and represent hydrogen or unsubstituted C₁-C₂ alkyl. Particularly preferred substituents include hydroxyl and —NR′R″ groups where R′ and R″ are the same and represent hydrogen.

When the alkyl, alkenyl, alkynyl and aliphatic groups above are substituted by two or three substituents, it is preferred that not more than two substituents are selected from hydroxyl. More preferably, not more than one substituent is selected from hydroxyl.

Most preferably, the alkyl, alkenyl and alkynyl groups above are unsubstituted.

As used herein, a C₁-C₆ alkyl group is a linear or branched alkyl group containing from 1 to 6 carbon atoms, for example a C₁-C₄ alkyl group containing from 1 to 4 carbon atoms, preferably a C₁-C₂ alkyl group containing from 1 to 2 carbon atoms. Examples of C_{I}-Cₐ alkyl groups include methyl, ethyl, n-propyl, i-propyl, n-butyl, i-butyl and t-butyl. For the avoidance of doubt, where two alkyl groups are present in a compound of the present invention, the alkyl groups may be the same or different.

As used herein, a C₂-C₆ alkenyl group is a linear or branched alkenyl group having at least one double bond of either cis or trans configuration where applicable and containing from 2 to 6 carbon atoms, for example a C₂-C₄ alkenyl group containing from 2 to 4 carbon atoms, such as —CH═CH₂ or —CH₂—CH═CH₂, —CH₂—CH₂—CH═CH₂, —CH₂—CH═CH—CH₃, —CH═C(CH₃)—CH₃ and —CH₂—C(CH₃)═CH₂, preferably a C₂ alkenyl group having 2 carbon atoms. For the avoidance of doubt, where two alkenyl groups are present in a compound of the present invention, they may be the same or different.

As used herein, a C₂-C₆ alkynyl group is a linear or branched alkynyl group containing from 2 to 6 carbon atoms, for example a C₂-C₄ alkynyl group containing from 2 to 4 carbon atoms, preferably a C₂ alkynyl group containing 2 carbon atoms. Exemplary alkynyl groups include —C≡H or —CH₂—C≡H, as well as 1- and 2-butynyl, 2-methyl-2-propynyl, 2-pentynyl, 3-pentynyl, 4-pentynyl, 2-hexynyl, 3-hexynyl, 4-hexynyl and 5-hexynyl. For the avoidance of doubt, where two alkynyl groups are present in a compound of the present invention, they may be the same or different.

Preferably, said C₁-C₆ alkyl group is a C₁-C₂ alkyl group, said C₂-C₆ alkenyl group is a C₂ alkenyl group and said C₂-C₆ alkynyl group is a C₂ alkynyl group.

As used herein, a halogen atom is chlorine, fluorine, bromine or iodine.

As used herein, a C₁-C₆ alkoxy group or C₂-C₆ alkenyloxy group is typically a said C₁-C₆ alkyl (e.g. a C₁-C₄ alkyl) group or a said C₂-C₆ alkenyl (e.g. a C₂-C₄ alkenyl) group respectively which is attached to an oxygen atom.

A haloalkyl, haloalkenyl, haloalkoxy or haloalkenyloxy group is typically a said alkyl, alkenyl, alkoxy or alkenyloxy group respectively which is substituted by one or more said halogen atoms. Typically, it is substituted by 1, 2 or 3 said halogen atoms. Preferred haloalkyl and haloalkoxy groups include perhaloalkyl and perhaloalkoxy groups, such as —CX₃ and —OCX₃ wherein X is a said halogen atom, for example chlorine and fluorine.

As used herein, a C₁-C₄ alkylthio or C₂-C₄ alkenylthio group is typically a said C₁-C₄ alkyl group or a C₂-C₄ alkenyl group respectively which is attached to a sulphur atom, for example —S—CH₃.

As used herein, a C₁-C₄ hydroxyalkyl group is a C₁-C₄ alkyl group substituted by one or more hydroxy groups. Typically, it is substituted by one, two or three hydroxy groups. Preferably, it is substituted by a single hydroxy group.

As used herein, a C₆-C₁₀ aryl group is a monocyclic or polycyclic, preferably monocyclic, aromatic ring containing from 6 to 10 carbon atoms, for example a C₆ aryl group containing 6 carbon atoms. Examples of such aryl groups include phenyl, naphthalene and azulene. Phenyl is preferred.

As used herein, a 5- to 10-membered heteroaryl group is a monocyclic or polycyclic, preferably monocyclic, 5- to 10-membered aromatic ring, such as a 5- or 6-membered ring, containing at least one heteroatom, for example 1, 2, 3 or 4 heteroatoms, selected from O, S and N. When the ring contains 4 heteroatoms these are preferably all nitrogen atoms. Examples include thienyl, furyl, pyrrolyl, imidazolyl, thiazolyl, isothiazolyl, pyrazolyl, oxazolyl, isoxazolyl, triazolyl, thiadiazolyl, oxadiazolyl, pyridinyl, pyridazinyl, pyrimidinyl, pyrazinyl, triazinyl and tetrazolyl groups. Thienyl, pyrrolyl, imidazolyl, thiazolyl, isothiazolyl, pyrazolyl, oxazolyl, isoxazolyl, triazolyl, pyridinyl, pyridazinyl, pyrimidinyl and pyrazinyl groups are preferred, e.g. pyrrolyl, imidazolyl, thiazolyl, isothiazolyl, pyrazolyl, oxazolyl, isoxazolyl, triazolyl, pyridinyl, pyridazinyl, pyrimidinyl and pyrazinyl groups. More preferred groups are thienyl, pyridinyl, pyridazinyl, pyrimidinyl, pyrazinyl, pyrrolyl and triazinyl, e.g. pyridinyl, pyridazinyl, pyrimidinyl, pyrazinyl, pyrrolyl and triazinyl, most preferably pyridinyl.

As used herein, a 5- to 10-membered heterocyclyl group is a non-aromatic, saturated or unsaturated monocyclic or polycyclic, preferably monocyclic, C₅₋₁₀ carbocyclic ring in which one or more, for example 1, 2, 3 or 4, of the carbon atoms are replaced with a moiety selected from N, O, S, S(O) and S(O)₂, and wherein one or more of the remaining carbon atoms is optionally replaced by a group —C(O)— or —C(S)—. When one or more of the remaining carbon atoms is replaced by a group —C(O)— or —C(S)—, preferably only one or two (more preferably two) such carbon atoms are replaced. Typically, the 5- to 10-membered heterocyclyl ring is a 5- to 6-membered ring.

Suitable heterocyclyl groups include azetidinyl, oxetanyl, thietanyl, pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, methylenedioxyphenyl, ethylenedioxyphenyl, thiomorpholinyl, S-oxo-thiomorpholinyl, S,S-dioxo-thiomorpholinyl, morpholinyl, 1,3-dioxolanyl, 1,4-dioxolanyl, trioxolanyl, trithianyl, imidazolinyl, pyranyl, pyrazolinyl, thioxolanyl, thioxothiazolidinyl, 1H-pyrazol-5-(4H)-onyl, 1,3,4-thiadiazol-2(3H)-thionyl, oxopyrrolidinyl, oxothiazolidinyl, oxopyrazolidinyl, succinimido and maleimido groups and moieties. Preferred heterocyclyl groups are pyrrolidinyl, imidazolidinyl, oxazolidinyl, isoxazolidinyl, thiazolidinyl, isothiazolidinyl, tetrahydrofuranyl, tetrahydrothienyl, tetrahydropyranyl, tetrahydrothiopyranyl, dithiolanyl, dioxolanyl, pyrazolidinyl, piperidinyl, piperazinyl, hexahydropyrimidinyl, thiomorpholinyl and morpholinyl groups and moieties.

For the avoidance of doubt, although the above definitions of heteroaryl and heterocyclyl groups refer to an “N” moiety which can be present in the ring, as will be evident to a skilled chemist the N atom will be protonated (or will carry a substituent as defined below) if it is attached to each of the adjacent ring atoms via a single bond.

As used herein, a C₃-C₇ carbocyclic group is a non-aromatic saturated or unsaturated hydrocarbon ring having from 3 to 7 carbon atoms. Preferably it is a saturated or mono-unsaturated hydrocarbon ring (i.e. a cycloalkyl moiety or a cycloalkenyl moiety) having from 3 to 7 carbon atoms, more preferably having from 3 to 6 carbon atoms. Examples include cyclopropyl, cyclobutyl, cyclopentyl and cyclohexyl and their mono-unsaturated variants, more particularly cyclopentyl and cyclohexyl. A C₃-C₇ carbocyclyl group also includes C₃-C₇ carbocyclyl groups described above but wherein one or more ring carbon atoms are replaced by a group —C(O)—. More preferably, 0, 1 or 2 ring carbon atoms (most preferably 0) are replaced by —C(O)—. Most preferably, said C₃-C₇ carbocyclyl group is cyclohexyl.

Typically the aryl, heteroaryl, heterocyclyl and carbocyclyl groups in R₁ and R₅ are unsubstituted or substituted by 1, 2, 3 or 4 unsubstituted substituents, for example by 1, 2 or 3 unsubstituted substituents. Preferred substituents include halogen atoms and C₁-C₄ alkyl, C₂-C₄ alkenyl, C₁-C₄ alkoxy, C₂-C₄ alkenyloxy, C₁-C₄ haloalkyl, C₂-C₄ haloalkenyl, C₁-C₄ haloalkoxy, C₂-C₄ haloalkenyloxy, hydroxyl, mercapto, cyano, nitro, C₁-C₄ hydroxyalkyl, C₂-C₄ hydroxyalkenyl, C₁-C₄ alkylthio, C₂-C₄ alkenylthio and —NR″R″ groups wherein each R′ and R″ is the same or different and represents hydrogen or C₁-C₄ alkyl. More preferred substituents include halogen atoms and unsubstituted C₁-C₄ alkyl, C₁-C₄ alkoxy, hydroxyl, C₁-C₄ haloalkyl, C₁-C₄ haloalkoxy, C₁-C₄hydroxyalkyl, cyano, nitro, —SR′ and —NR′R″ groups where R′ and R″ are the same or different and represent hydrogen or unsubstituted C₁-C₂ alkyl. More preferred substituents include halogen atoms, hydroxyl groups and C₁-C₂ alkyl and C₁-C₂ alkoxy groups.

Most preferably, the aryl, heteroaryl, heterocyclyl and carbocyclyl groups above are unsubstituted.

When the aryl, heteroaryl, heterocyclyl and carbocyclyl groups in R₁ and R₅ are substituted by two, three or four substituents, it is preferred that not more than two substituents are selected from hydroxyl, cyano and nitro. More preferably, not more than one substituent is selected from hydroxyl, cyano and nitro.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulphonic, ethanesulphonic, benzenesulphonic or p-toluenesulphonic acid. Pharmaceutically acceptable bases include alkali metal (e.g. sodium or potassium) and alkali earth metal (e.g. calcium or magnesium) hydroxides and organic bases such as alkyl amines, aralkyl amines and heterocyclic amines.

The term “solvate” refers to a complex or aggregate formed by one or more molecules of a solute, i.e. compounds of the invention or pharmaceutically-acceptable salts thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

The compounds of the invention contain a chiral center. Accordingly, they can be used in the form of a racemic mixture, an enantiomer, or a mixture enriched in one or more stereoisomer. The scope of the invention as described and claimed encompasses the racemic forms of the compounds of the invention as well as the individual enantiomers, and stereoisomer-enriched mixtures.

It will be appreciated that the term “or a pharmaceutically acceptable salt or solvate thereof” is intended to include all permutations of salts and solvates, such as solvates of pharmaceutically-acceptable salts of compounds of the invention.

R₅ and R₆ may be an aliphatic group having 3 to 29 carbon atoms. Typically, the aliphatic group is not cyclic. The aliphatic group is typically linear or branched, preferably linear. Typically the aliphatic group has 7 to 25 carbon atoms, more preferably 11 to 25 carbon atoms. The aliphatic group is typically unsubstituted or substituted with one hydroxyl group. The aliphatic group is preferably unsubstituted.

Aliphatic groups may be saturated, monounsaturated or polyunsaturated. Saturated aliphatic groups are preferred.

Typically, saturated aliphatic groups have from 7 to 25 carbon atoms, preferably 11 to 17 carbon atoms.

Monounsaturated aliphatic groups typically contain a single C═C double bond. The double bond has cis or trans configuration. The single double bond may be present at any point in the aliphatic group, but is typically 7 or 9 carbon atoms from the end of the aliphatic group distal to the (C═O) group to which the aliphatic group is attached. Typically, monounsaturated aliphatic groups have from 7 to 25 carbon atoms, preferably 15 to 23 carbon atoms.

Polyunsaturated aliphatic groups typically contain two or more C═C double bonds, for example 2, 3, 4, 5 or 6 C═C double bonds. Each double bond may have cis or trans configuration. The double bonds may be present at any point in the aliphatic chain, but typically, the C═C double bond furthest from the (C═O) group to which the aliphatic group is attached is 3, 6 or 9 carbon atoms from the end of the aliphatic group distal to the (C═O) group to which the aliphatic group is attached. Typically, polyunsaturated aliphatic groups have from 7 to 25 carbon atoms, preferably 15 to 23 carbon atoms.

Typically, said aliphatic group is the group R, wherein R—CO₂H is a fatty acid. Preferably, said fatty acid is lauric acid, myristic acid, palmitic acid, stearic acid palmitoleic acid, cis-vaccenic acid, oleic acid, eicosenoic acid, erucic acid, nervonic acid, alpha-linolenic acid, stearidonic acid, eicosatrienoic acid, eicosatetraenoic acid, eicosapentaenoic acid, docosapentaenoic acid, docosahexaenoic acid, tetracosapentaenoic acid, tetracosahexaenoic acid, linoleic acid, gamma-linolenic acid, eicosadienoic acid, dihommo-gamma-linolenic acid, arachidonic acid, docosadienoic acid, adrenic acid, docosapentaenoic acid, or mead acid. More preferably, said fatty acid is lauric acid, myristic acid, palmitic acid, or stearic acid.

In one embodiment, the aliphatic group having 3 to 29 carbon atoms is the aliphatic group of a PUFA derivative of formula (I) as defined herein, i.e. the aliphatic group is of formula —(CH₂)₃-Alk-(CH₂)₄CH₃, wherein -Alk- is as defined herein.

In a preferred embodiment, the aliphatic group having 3 to 29 carbon atoms is the aliphatic group of 13-hydroxyoctadecadienoic acid or 15-hydroxyeicosatrienoic acid, i.e. the aliphatic group is —(CH₂)₇-[cis]CH═CH-[trans]CH═CH—CH(OH)—(CH₂)₄CH₃, or —(CH₂)₆-[cis]CH═CH—CH₂-[cis]CH═CH-[trans]CH═CH—CH(OH)—(CH₂)₄CH₃.

In a more preferred embodiment, the PUFA derivative of formula (I) is of formula R′O—CH₂—CH(OR′)—CH₂—OR′, wherein each R′ is the same or different and is the aliphatic group of 13-hydroxyoctadecadienoic acid or 15-hydroxyeicosatrienoic acid, i.e. R′ is —(CH₂)₇-[cis]CH═CH—[trans]CH═CH—CH(OH)—(CH₂)₄CH₃, or —(CH₂)₆-[cis]CH═CH—CH₂-[cis]CH═CH-[trans]CH═CH—CH(OH)—(CH₂)₄CH₃. Preferably each R′ is the same. Thus, the PUFA derivative of formula (I) is preferably

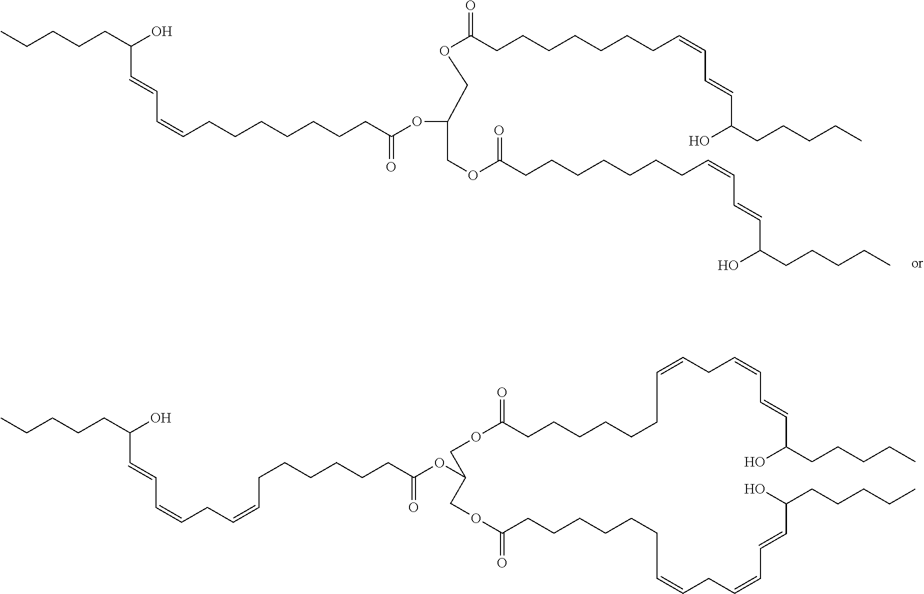

It is to be understood that the left hand side of the -Alk-moiety is bonded to the unsaturated carbon chain bearing the —COOR₁ moiety and the right hand side of the -Alk-group is bonded to the saturated carbon chain.

R₁, R₂, and R₅ may be “drug moieties”. Typically, the “drug moiety” is a drug moiety that is effective in treating neuropathy, neuropathic pain and/or diabetic neuropathy. Suitable such drug moieties are well known in the art.

When R₁ is a drug moiety, the drug moiety may be bonded to the oxygen atom directly or indirectly, preferably directly. When R₂ is a drug moiety, the drug moiety may be bonded to the oxygen atom directly or indirectly, preferably directly. Direct linkage to said oxygen atoms may occur through any convenient functional group on the drug moiety, such as a carboxy group.

When R₅ is a drug moiety, the drug moiety may be bonded to the carboxyl group directly or indirectly, preferably directly. Direct linkage to said carboxy group may occur through any convenient functional group on the drug moiety, such as a hydroxyl group or amino group.

Indirect linkage will occur through a linking moiety. The person skilled in the art is well aware of suitable linking moieties. Suitable linking moieties include bi- and multi-functional alkyl, aryl, aralkyl or peptidic moieties.

Typically, the drug moiety is an aldose reductase inhibitor, an ACE inhibitor, a vitamin or an anti-oxidant. Typically, the drug moiety is buprenorphine, cannabidiol, tetrahydrocannabinol, duloxetine, epalrestat, lidocaine, pregabalin, varicella zoster virus, alprostadil, lacosamide, transacin, mexiletine, acetyl-L-carnitine, amitriptyline, ketamine, desvenlafaxine, dextromethorphan, fidarestat, gabapentin, GW-1000 (GW Pharmaceuticals), lamotigrine, memantine, NGX-4010 (NeurogesX), ranirestat, ruboxistaurin, 681323 (GSK), ABT 894 PII NP (Abbott/NeuroSearch), ADL 5859 (Adolor/Pfizer), ajulemic acid, an alpha adrenergic agonist, beraprost, bicifadine, brivaracetam, bupivacaine, BVT 115959 (Biovitrum), candesartan cilexetil, cannabinor, CNS 5161 (CeNeS), coleneuramide, davasaicin, galantamine, FARBETIC, CNSB 001 (CNSBio), gabapentin enacarbil, VEGF ZFP (Sangamo BioSciences), ibudilast, indantadol, KD 7040 PII NP (Kalypsys), lidorestat MK 0759 (Merck & Co), perampanel, proinsulin C-peptide, QR 333 (Quigley), radiprodil, ralfinamide, REN 1654 (Evotec), SLC 022 (Solace), S,S-reboxetine, SSR 180575 (Sanofi-Aventis), TAK 428 (Takeda), timcodar, transacin, TRO 19622 (Trophos), transdur bupivacaine, vitamin B1, vitamin B12, or lipoic acid. Preferably, the drug moiety is pregabilin, carbamezapine, lidocaine, gabapentin or cymbalta.

When there is more than one drug moiety present in the compound of formula (I), each drug moiety may be the same or different. Typically, compounds of formula (I) which comprise a drug moiety comprise only one such drug moiety.

Typically, -Alk- is —(CH₂)₄-[cis]CH═CH-[trans]CH═CH—CH(OR₂)— or —(CH₂)₃-[cis]CH═CH—CH₂-[cis]CH═CH-[trans]CH═CH—CH(OR₂)—, wherein each R₂ is the same or different and is as defined herein.

Preferably, -Alk- is —(CH₂)₃-[cis]CH═CH—CH₂-[cis]CH═CH-[trans]CH═CH—CH(OR₂)—, wherein R₂ is as defined herein.

Typically, R₁ is not a drug moiety.

Typically, R₁ is a hydrogen atom; or R₁ is a C₁-C₄ alkyl, C₂-C₄ alkenyl, C₂-C₄ alkynyl, C₆ aryl, 5- to 6-membered heteroaryl, C₃-C₆ carbocyclyl or 5- to 6-membered heterocyclyl group; or R₁ is a group of formula —CH₂—CH(OR₃)—CH₂—(OR₄), wherein R₃ and R₄ are as defined herein; or R₁ is a group of formula —(CH₂OCH₂)ₘOH, wherein m is as defined herein, wherein said alkyl, alkenyl and alkynyl groups are the same or different and are each unsubstituted or substituted with 1, or 2 unsubstituted substituents which are the same or different and are selected from halogen atoms, C₁-C₄ alkoxy, hydroxyl, and —NR′R″ groups where R′ and R″ are the same or different and represent hydrogen or unsubstituted C₁-C₂ alkyl; and said aryl, heteroaryl, carbocyclyl and heterocyclyl groups are the same or different and are each unsubstituted or substituted by 1, 2 or 3 unsubstituted substituents which are the same or different and are selected from halogen atoms, and cyano, nitro, C₁-C₄ alkyl, C₁-C₄ alkoxy, and —NR″R″ groups wherein each R′ and R″ is the same or different and represents hydrogen or unsubstituted C₁-C₂ alkyl group.

Preferably, R₁ is a hydrogen atom; or R₁ is an unsubstituted C₁-C₄ alkyl group; or R₁ is a group of formula —CH₂—CH(OR₃)—CH₂—(OR₄), wherein R₃ and R₄ are as defined herein; or R₁ is a group of formula —(CH₂OCH₂)ₘOH, wherein m is as defined herein.

More preferably, R₁ is a hydrogen atom; or R₁ is a group of formula —CH₂—CH(OR₃)—CH₂—(OR₄), wherein R₃ and R₄ are as defined herein, and wherein at least one of R₃ or R₄ is —(C═O)—R₆, wherein R₆ is as defined herein.

Most preferably, R, is a hydrogen atom.

m is typically an integer of from 5 to 150, preferably from 10 to 50.

R₃ is typically —(C═O)—R₆, wherein R₆ is as defined herein.

R₄ is typically —(C═O)—R₆, wherein R₆ is as defined herein.

Preferably, both R₃ and R₄ are —(C═O)—R₆, wherein each R₆ may be the same or different and is as defined herein.

Typically, when R₃ and R₄ are both —(C═O)—R₆, then R₅ is not an aliphatic group having 3 to 29 carbon atoms.

R₆ is an aliphatic group having from 3 to 29 carbon atoms, as defined herein. Typically, said aliphatic group is saturated. Typically, R₆ is an aliphatic group having 7 to 25 carbon atoms, preferably 11 to 17 carbon atoms. Preferably, R₆ is a group R, wherein R—CO₂H is auric acid, myristic acid, palmitic acid, or stearic acid.

Typically, R₂ is not a drug moiety.

Typically, R₂ is a hydrogen atom; or R₂ is a group —(C═O)—R₅, wherein R₅ is a C₁-C₄ alkyl, C₂-C₄ alkenyl, C₂-C₄ alkynyl, C₆ aryl, 5- to 6-membered heteroaryl, C₃-C₆ carbocyclyl or 5- to 6-membered heterocyclyl group, or R₅ is an aliphatic group having from 3 to 29 carbon atoms; or R₂ is a group of formula —(CH₂OCH₂)ₙOH, wherein n is as defined herein, wherein said alkyl, alkenyl and alkynyl groups are the same or different and are each unsubstituted or substituted with 1, or 2 unsubstituted substituents which are the same or different and are selected from halogen atoms, C₁-C₄ alkoxy, hydroxyl, and —NR′R″ groups where R′ and R″ are the same or different and represent hydrogen or unsubstituted C₁-C₂ alkyl; and said aryl, heteroaryl, carbocyclyl and heterocyclyl groups are the same or different and are each unsubstituted or substituted by 1, 2 or 3 unsubstituted substituents which are the same or different and are selected from halogen atoms, and cyano, nitro, C₁-C₄ alkyl, C₁-C₄ alkoxy, and —NR′R″ groups wherein each R′ and R″ is the same or different and represents hydrogen or unsubstituted C₁-C₂ alkyl group.

Preferably, R₂ is a hydrogen atom; or R₂ is a group —(C═O)—R₅, wherein R₅ is unsubstituted C₁-C₄ alkyl; or R₂ is a group —(C═O)—R₅, wherein R₅ is an aliphatic group having from 3 to 29 carbon atoms; or R₂ is a group of formula —(CH₂OCH₂)ₙOH, wherein n is as defined herein.

More preferably, R₂ is a hydrogen atom; or R₂ is a group —(C═O)—R₅, wherein R₅ is an aliphatic group having from 3 to 29 carbon atoms; or R₂ is a group of formula —(CH₂OCH₂)ₙOH, wherein n is as defined herein.

Most preferably, R₂ is a hydrogen atom.

n is typically an integer of from 5 to 150, preferably from 10 to 50.

When R₅ is an aliphatic group having 3 to 29 carbon atoms, said aliphatic group is as defined herein. Typically, said aliphatic group is saturated. Typically, R₅ is an aliphatic group having 7 to 25 carbon atoms, preferably 11 to 17 carbon atoms. Preferably, R₅ is a group R, wherein R—CO₂H is auric acid, myristic acid, palmitic acid, or stearic acid.

In a preferred embodiment, -Alk- is —(CH₂)₄-[cis]CH═CH-[trans]CH═CH—CH(OR₂)— or —(CH₂)₃-[cis]CH═CH—CH₂-[cis]-CH═CH-[trans]CH═CH—CH(OR₂)—; R₁ is a hydrogen atom, an unsubstituted C₁-C₄ alkyl group, or a group of formula —CH₂—CH(OR₃)—CH₂—(OR₄), wherein R₃ and R₄ are each independently hydrogen atoms or —(C═O)—R₆, wherein R₆ is a linear aliphatic group having from 11 to 25 carbon atoms, which aliphatic group is unsubstituted or substituted with one hydroxyl group, or R₁ is a group of formula —(CH₂OCH₂)ₘOH, wherein m is an integer of from 5 to 150; and each R₂ is the same or different and is a hydrogen atom; a group —(C═O)—R₅, wherein R₅ is unsubstituted C₁-C₄ alkyl, or a group —(C═O)—R₅, wherein R₅ is a linear aliphatic group having from 11 to 25 carbon atoms, which aliphatic group is unsubstituted or substituted with one hydroxyl group; or a group of formula —(CH₂OCH₂)ₙOH, wherein n is an integer of from 5 to 150.

In a more preferred embodiment, -Alk- is —(CH₂)₄-[cis]CH═CH-[trans]CH═CH—CH(OR₂)— or —(CH₂)₃-[cis]CH═CH—CH₂-[cis]CH═CH-[trans]CH═CH—CH(OR₂)—; R₁ is a hydrogen atom, a group of formula —CH₂—CH(OR₃)—CH₂—(OR₄), wherein R₃ and R₄ are each independently hydrogen atoms or —(C═O)—R₆, wherein R₆ is an unsubstituted linear, saturated aliphatic group having from 11 to 17 carbon atoms, and wherein at least one of R₃ or R₄ is —(C═O)—R₆; and each R₂ is the same or different and is a hydrogen atom; a group —(C═O)—R₅, wherein R₅ is an unsubstituted linear, saturated aliphatic group having from 11 to 17 carbon atoms; or a group of formula —(CH₂OCH₂)ₙOH, wherein n is an integer of from 10 to 50.

Typically, both R₁ and R₂ are hydrogen atoms.

In an even more preferred embodiment, Alk- is —(CH₂)₄-[cis]CH═CH-[trans]CH═CH—CH(OR₂)— or —(CH₂)₃-[cis]CH═CH—CH₂-[cis]CH═CH-[trans]CH═CH—CH(OR₂)—; R₁ is a hydrogen atom and R₂ is a hydrogen atom.

In a particularly preferred embodiment, -Alk- is —(CH₂)₃-[cis]CH═CH—CH₂-[cis]CH═CH-[trans]CH═CH—CH(OR₂)—, and R₁ and R₂ are both hydrogen atoms. In this embodiment, the PUFA derivative of formula (I) is 15-HETrE and is represented by the formula

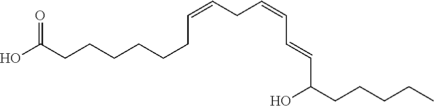

In another embodiment, -Alk- is —(CH₂)₄-[cis]CH═CH-[trans]CH═CH—CH(OR₂)—, and R₁ and R₂ are both hydrogen atoms. In this embodiment, the PUFA derivative of formula (I) is 13-HODE and is represented by the formula

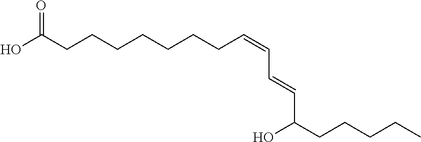

In one embodiment, the PUFA derivative of formula (I) is present as a racemic mixture of the R and S enantiomers.

In another embodiment, the PUFA derivative of formula (I) is present as the R enantiomer.

In another embodiment, the PUFA derivative of formula (I) is present as the S enantiomer.

Typically, the mammal is a human.

Typically, use of the invention involves administering compounds orally, parenterally or intravenously. Oral administration is preferred.

When the use of the invention involves administering a compound parenterally or intranvenously, the compound is typically a salt or solvate of a PUFA derivative of formula (I), as defined herein.

Typically, use of the invention involves administering compounds as one or more treatments per day, preferably from 1 to 4 treatments per day, more preferably from 1 to 2 treatments per day.

Typically, use of the invention involves administering compounds at a daily dosage of from 1 μg/kg/day to 100 mg/kg/day, preferably from 10 82 g/kg/day to 50 mg/kg/day, more preferably from 50 μg/kg/day to 10 mg/kg/day, most preferably from 0.1 mg/kg/day to 5 mg/kg/day.

Typically, the use of the invention involves treating nerve damage, preferably peripheral neuropathy, more preferably peripheral neuropathy caused by metabolic and/or endocrine disorders, most preferably diabetic neuropathy, in a mammal, preferably a human.

In one embodiment, the nerve damage is nerve damage caused by vaso-occlusive crises resulting from sickle cell anaemia.

In one embodiment, the nerve damage is nerve damage to the central nervous system. Thus, in this embodiment, compounds of the invention are for use in treating and/or preventing disorders of the central nervous system including Alzheimer's disease, Parkinson's disease and/or dementia.

In a preferred embodiment, the nerve damage is nerve damage to the peripheral nervous system, i.e. compounds of the invention are for use in treating and/or preventing peripheral neuropathy.

Peripheral neuropathy is typically peripheral neuropathy resulting from genetic diseases, metabolic and/or endocrine disorders, toxic causes, fluoroquinoline toxicity syndrome, inflammatory diseases, vitamin deficiency, physical trauma, shingles, malignant diseases, HIV/AIDS, radiation and/or chemotherapy.

The genetic diseases mentioned-above include Friedreich's ataxia and Charcot-Marie-Tooth syndrome. The metabolic and/or endocrine discorders mentioned above include diabetes mellitus, chronic renal failure, porphryia, amyloidosis, liver failure and hyperthyroidism. The toxic causes mentioned above include alcoholism, drug toxicity (e.g. vincristine, phenytoin, isoniazid), organic metal poisoning, heavy metal poisoning, and excess Vitamin B6 intake. The inflammatory diseases mentioned above include Guillain-Barre syndrome, systemic lupus erythematosis, leprosy, Sjogren's syndrome. The vitamin deficiency mentioned above includes vitamin B12, vitamin A, vitamin E and vitamin B1 deficiency. The physical trauma mentioned above includes compression, pinching, and cutting of nerves and also includes damage caused by strokes.

Typically, the peripheral neuropathy is peripheral neuropathy caused by metabolic and/or endocrine disorders. Preferably, the peripheral neuropathy is diabetic neuropathy.

As used herein, diabetes includes both type I and type II diabetes.

Typically, the diabetic neuropathy is diabetic neuropathy of the sensory nerves, motor nerves and/or autonomic nerves.

In one embodiment, the diabetic neuropathy is cranial neuropathy, or diabetic third nerve palsy.

It is a finding that compounds of formula (I) improve nerve function. Accordingly, the present invention provides use of compounds, as defined herein, in the manufacture of a medicament for use in improving nerve function in a mammal. Typically, said mammal is suffering from neuropathy, in particular diabetic neuropathy. The present invention also provides use of compounds, as defined herein in the manufacture of a medicament for use in treating or preventing neuropathy, in particular diabetic neuropathy, in a mammal by improving nerve function.

The present invention also provides use of compounds, as defined herein, in the manufacture of a medicament for use in treating or preventing numbness, dysesthesia, dysphagia, speech impairment, tremor, muscle weakness, dizziness, tiredness, heaviness, drooping of the face, mouth or eyelid, vision changes, loss of balance, gait abnormalities, tingling, burning sensations, pain (including pain caused by sickle cell anaemia), in particular burning, stabbing and electric shock like pain, itching, crawling sensations, pins and needles, tingling in the legs and feet, decreased or loss of temperature perception, decreased or loss of ankle reflex, decreased or loss of sensitivity to vibrations, cramps, fasciculations, foot sores, muscle wastage, in particular of the thigh muscles, abnormal blood pressure and heart rate, reduced ability to perspire, gustatory sweating, indigestion, constipation, diarrhea, bladder dysfunction, incontinence, bladder infections, impotence, and sexual dysfunction (e.g. erectile dysfunction), arising from diabetic neuropathy. Treating or preventing dizziness, indigestion, bladder infections, foot sores, wastage of thigh muscles, sexual disfunction (e.g. erectile dysfunction), numbness, burning sensations, pain, tingling in the legs and feet, decreased or loss of temperature perception, decreased or loss of ankle reflex and/or decreased or loss of sensitivity to vibrations, arising from diabetic neuropathy is preferred. Treating sexual dysfunction, in particular erectile dysfunction, is more preferred.

Typically, the use of the invention involves co-administering compounds, as defined herein, with one or more further therapeutic agents. Said further therapeutic agents are typically effective in treating diabetes, neuropathy, neuropathic pain and/or diabetic neuropathy. Such therapeutic agents are well known to the skilled person and include, but are not limited to, aldose reductase inhibitors, ACE inhibitors, vitamins and anti-oxidants. Suitable further therapeutic agents include buprenorphine, cannabidiol, tetrahydrocannabinol, duloxetine, epalrestat, lidocaine, pregabalin, varicella zoster virus, alprostadil, lacosamide, transacin, mexiletine, acetyl-L-carnitine, amitriptyline, ketamine, desvenlafaxine, dextromethorphan, fidarestat, gabapentin, GW-1000 (GW Pharmaceuticals), lamotigrine, memantine, NGX-4010 (NeurogesX), ranirestat, ruboxistaurin, 681323 (GSK), ABT 894 PII NP (Abbott/NeuroSearch), ADL 5859 (Adolor/Pfizer), ajulemic acid, an alpha adrenergic agonist, beraprost, bicifadine, brivaracetam, bupivacaine, BVT 115959 (Biovitrum), candesartan cilexetil, cannabinor, CNS 5161 (CeNeS), coleneuramide, davasaicin, galantamine, FARBETIC, CNSB 001 (CNSBio), gabapentin enacarbil, VEGF ZFP (Sangamo BioSciences), ibudilast, indantadol, KD 7040 PII NP (Kalypsys), lidorestat MK 0759 (Merck & Co), perampanel, proinsulin C-peptide, QR 333 (Quigley), radiprodil, ralfinamide, REN 1654 (Evotec), SLC 022 (Solace), S,S-reboxetine, SSR 180575 (Sanofi-Aventis), TAK 428 (Takeda), timcodar, transacin, TRO 19622 (Trophos), transdur bupivacaine, vitamin B1, vitamin B12, and lipoic acid. The appropriate dosages of the one or more further therapeutic agents for coadministration with the compounds, as defined herein, will be evident to the person skilled in the art.

The compounds used in the invention are typically commercially available, or may be prepared by analogy with known methods. Thus, 9-HODE, 13-HODE, 5-HETrE, 8-HETrE and 15-HETrE are all commercially available (Cayman Chemicals). These available fatty acids can easily be derivatised to obtain PUFA derivatives of formula (I) by known methods.

For example, PUFA derivatives of formula (I) as defined herein, wherein R₁ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group; or R₁ is a group of formula —CH₂—CH(OR₃)—CH₂—(OR₄), wherein R₃ and R₄ are as defined herein; or R₁ is a group of formula —(CH₂OCH₂)ₘOH, wherein m is as defined herein, can be prepared by esterifying a compound of formula

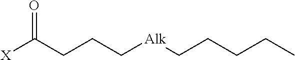

wherein -Alk- is as defined herein and X is a leaving group, for example a halogen atom, a tosylate or mesylate group with an alcohol of formula R₁′—OH, wherein R₁′ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group; or R₁′ is a group of formula —CH₂—CH(OR₃)—CH₂—(OR₄), wherein R₃ and R₄ are as defined herein; or R₁′ is a group of formula —(CH₂OCH₂)ₘOH, wherein m is as defined herein, to obtain a PUFA derivative of formula (I) as defined herein. Alternatively, X may be a hydroxyl group. In that case, the reaction is preferably carried out under acidic conditions, or in the presence of a suitable catalyst, for example pyridine. Compounds of formula R₁′—OH are typically commercially available or may be prepared by analogy with known methods.

PUFA derivatives of formula (I) as defined herein, wherein R₂ is a group —(C═O)—R₅, wherein R₅ is a C₁-C₆ alkyl, C₂-C₆ alkenyl, C₂-C₆ alkynyl, a C₆-C₁₀ aryl, a 5- to 10-membered heteroaryl, a C₃-C₇ carbocyclyl or a 5- to 10-membered heterocyclyl group, or R₅ is an aliphatic group having from 3 to 29 carbon atoms, can be prepared by treating a PUFA derivative of formula (I), as defined herein, wherein R₂ is hydrogen, with a carboxylic acid derivative Y—(C═O)—R′₅, wherein R′₅ is a C₁-C₆ alkyl, C₂-C₆ alkenyl, C₂-C₆ alkynyl, a C₆-C₁₀ aryl, a 5- to 10-membered heteroaryl, a C₃-C₇ carbocyclyl or a 5- to 10-membered heterocyclyl group, or R′₅ is an aliphatic group having from 3 to 29 carbon atoms, and Y is a leaving group, for example a halogen atom, a tosylate or mesylate group. Compounds of formula Y—(C═O)—R′₅ are typically commercially available or may be prepared by analogy with known methods.

PUFA derivatives of formula (I) as defined herein, wherein R₂ is a group of formula —(CH₂OCH₂)ₙOH, wherein n is as defined herein, can be prepared by treating a PUFA derivative of formula (I), as defined herein, wherein R₂ is hydrogen, with a compound of formula Z—(CH₂OCH₂)ₙOH, wherein n is as defined herein and Z is a good leaving group, for example a halogen atom, a tosylate or mesylate group. Compounds of formula Z—(CH₂OCH₂)ₙOH are typically commercially available or may be prepared by analogy with known methods.

PUFA derivatives of formula (I) as defined herein, wherein R₁ is a drug moiety as defined herein, can be prepared by treating a PUFA derivative of formula

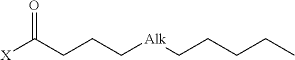

wherein -Alk- is as defined herein and X is a leaving group, for example a halogen atom, a tosylate or mesylate group with (a) a drug which comprises a nucleophilic group capable of reacting with the group X(C═O) of the PUFA derivative above, or (b) a drug which is linked to a linker moiety, as defined above, which linker moiety comprises a nucleophilic group capable of reacting with the group X(C═O) of the PUFA derivative above. Examples of such groups capable of reacting with the group X(C═O) of the PUFA derivative above include hydroxyl and amino groups.

PUFA derivatives of formula (I) as defined herein, wherein R₂ is a drug moiety as defined herein, can be prepared by treating a PUFA derivative of formula (I), as defined herein, wherein R₂ is hydrogen, with (a) a drug which comprises an electrophilic group capable of reacting with a hydroxyl group on the PUFA derivative, or (b) a drug which is linked to a linker moiety, as defined above, which linker moiety comprises an electrophilic group capable of reacting with a hydroxyl group on the PUFA derivative. Examples of such groups capable of reacting with a hydroxyl group include acid chlorides and alkyl halides.

PUFA derivatives of formula (I) as defined herein, wherein R₂ is a group —(C═O)R₅, wherein R₅ is a drug moiety as defined herein, can be prepared by treating a PUFA derivative of formula (I), as defined herein, wherein R₂ is hydrogen, with a carboxylic acid derivative Y—(C═O)—R″₅, wherein R″₅ is a drug moiety as defined herein, and Y is a leaving group, for example a halogen atom, a tosylate or mesylate group. Compounds of formula Y—(C═O)—R″₅ are typically commercially available or may be prepared by analogy with known methods.

The present invention also provides pharmaceutical compositions comprising compounds, as defined herein, of the invention and pharmaceutically acceptable diluents or carriers, for use in a method of treating or preventing nerve damage, as defined herein in a mammal, as defined herein. Preferred pharmaceutical compositions are sterile and pyrogen free.

The carrier is typically a mono-, di- or triglyceride oil. The carrier typically comprises corn, sunflower, safflower, cottonseed, grape seed, olive, evening primrose, borage, fish body or fish liver oil, or an ester of a fatty acid containing 16-26 carbon atoms and one or more double bonds. Said ester is typically an ethyl-eicosapentaenoic (ethyl-EPA), oleic, linoleic, alpha-linoleic, stearidonic, gamma-linolenic, dihommogammalinolenic, arachidonic, docosapentaenoic, or docosahexaenoic acid ester.

The pharmaceutical composition typically further comprises a fat-soluble antioxidant such as ascorbyl palmitate, tocopherol and/or ascorbic acid in the presence of lecithin.

The pharmaceutical composition typically further comprises an additive selected from aggregants, disaggregants, osmotic pressure regulating salts, buffers, sweeteners and colouring agents.

The pharmaceutical composition is typically administered in the form of a diatetic composition, or as a formulation selected from tablets, dragees, capsules, granules, suppositories, solutions, suspensions and lyophilized compositions.

When the pharmaceutical composition is in the form of a solution, the composition typically comprises a salt or solvate of a PUFA derivative of formula (I), as defined herein, and water.

When the pharmaceutical composition is in the form of a suspension, the composition typically comprises a compound of the present invention, as defined herein, water and one or more surfactants, such as Cremopohor or polysorbate.

Typically, pharmaceutical compositions of the present invention further comprise one or more additional therapeutic agents as defined herein. The amount of the one or more further therapeutic agents present in the composition will be evident to the person skilled in the art.

The present invention also provides a compound, as defined herein, for use in a method of treating or preventing nerve damage, as defined herein, in a mammal, as defined herein.

The present invention also provides a medicament comprising one or more compounds, as defined here, for use in a method of treating or preventing nerve damage, as defined herein, in a mammal, as defined herein. The medicament is typically formulated in the form of a pharmaceutical composition, as defined above.

The present invention also provides a compound, as defined herein, in substantially pure form or in association with one or more pharmaceutically acceptable diluents or carriers for use in a method of treating or preventing nerve damage, as defined herein, in a mammal, as defined herein. The one or more pharmaceutically acceptable diluents or carriers are typically as defined above.

As used herein, the term “substantially pure form” typically refers to a compound at a purity of 50% or greater, preferably 75% or greater, more preferably 90% or greater, even more preferably 95% or greater, and most preferably 99% or greater.

The present invention also provides a method of treating or preventing nerve damage, as defined herein, in a mammal, as defined herein, which method comprises administering to said mammal a therapeutically effective amount of a compound which is a PUFA derivative of formula (I) as defined herein or a pharmaceutically acceptable salt, or solvate thereof.

### EXAMPLES

All experiments were performed in accordance with regulations specified by the United Kingdom “Animal Procedures Act, 1986” and the National Institutes of Health “Principles of Laboratory Animal Care, 1985 revised version”.

### Example 1

Diabetes Induction and Treatment

Male Sprague-Dawley rats (Aberdeen University colony) were used, which were 19 weeks old at the start of the study. Diabetes was induced by intraperitoneal streptozotocin injection at 40-45 mg kg⁻¹ freshly dissolved in sterile 0.9% saline solution. This was verified 24 hours later by estimating hyperglycaemia (blood glucose>19.9 mM) and glycosuria, and diabetic state was monitored weekly using test strips for blood (tail vein) and urine glucose levels. Body weight was also monitored daily to check against body weight gain (which would indicate partial recovery of beta cell function and exclude diabetic status).

After 6 weeks of untreated diabetes, four experimental groups (n=6 per group) were treated for a 2-week period, with a daily oral administration of 13-HODE at a range of dosages (13-Hydoxydienoic acid, Equateq, Isle of Lewis, UK), added to the food and dispersed in a sunflower oil vehicle. Experimental groups were treated with doses of a representative compound of the invention,13-HODE, ranging from 0.01 mg/kg/day to 100 mg/kg/day.

### Nerve Conduction Velocity

Rats were anaesthetized with thiobutabarbital sodium (50-100 mg kg⁻¹ i.p.). The trachea was cannulated for artificial respiration.

The sciatic nerve was exposed between the sciatic notch and knee, and Motor nerve conduction velocity (NCV) was measured using concentric bipolar electrodes, as described in Cameron N E, et al (1989) Q J Exp Physiol 74:917-926 and Cameron N E, et al (1991) Diabetes 40:532-539, in the nerve branch to tibialis anterior muscle, which is representative of the whole sciatic nerve in terms of susceptibility to diabetes and treatment effects. Evoked electromyographic (EMG) potentials from each stimulating site were averaged 8 times, and Motor NCV was calculated by dividing the distance between stimulating electrodes by the average latency difference between the onset of EMG potentials evoked from the 2 sites. Nerve temperatures were monitored by thermocouple probes, and maintained in the range 36-38° C. by radiant heat. Body temperature was also maintained around 37° C. using a heated blanket.

Dose response curves for Example 1 are shown as FIGS. 1 and 2. A comparison of motor nerve conduction velocities in non-diabetic rats, diabetic rats and diabetic rats treated with 13-HODE is shown as FIG. 3.

NCV is a useful measure of nerve function in the peripheral nervous system and is a biomarker for peripheral neuropathy and, in particular, diabetic neuropathy. Patients suffering from diabetic neuropathy have lower NCV values than would be expected in normal, healthy patients. In rats, an NCV of 60 m/s is typical of a normal, healthy rat.

An NCV of 50 m/s is typical of a rat suffering from diabetic neuropathy. It can be seen that administration of 13-HODE results in a clear improvement of motor NCV in rats from an expected value for diabetic rats (around 50m/s) to that expected in non-diabetic rats (around 60 m/s).

### Example 2

An experiment was carried out in a similar fashion to Example 1 except that Sensory NCV was measured in the saphenous nerve between groin and mid calf. Direct nerve evoked potentials were recorded at the ankle using a unipolar platinum hook electrode.

A dose response curve for Example 2 is shown as FIG. 4. A comparison of sensory nerve conduction velocities in non-diabetic rats, diabetic rats and diabetic rats treated with 13-HODE is shown as FIG. 5.

It can be seen that administration of 13-HODE results in a clear improvement of sensory NCV in rats from an expected value for diabetic rats (around 50 m/s) to that expected in non-diabetic rats (around 60 m/s).

### Example 3

Experiments were carried out in similar fashions to Examples 1 and 2 except that 15-HETrE was used instead of 13-HODE.

The results of Example 3 are shown as FIG. 6.

It can be seen that administration of 15-HETrE results in a clear improvement of motor and sensory NCV in rats from an expected value for diabetic rats (around 50m/s) to that expected in non-diabetic rats (around 60 m/s).

### Example 4

Diabetes Induction and Treatment

Diabetes was induced in mature (19 week old) male Sprague-Dawley by streptozotocin injection (40-45 mg/kg i.p.). The diabetic state was monitored weekly using commercially available test strips for blood (tail vein) and urine glucose levels. Body weight would also be monitored daily. The criteria for the diabetic state are; blood glucose>19.9 mM, glycosuria, and no evidence of body weight gain (which would indicate partial recovery of beta cell function). At the end of the experiments, blood samples would be taken for the determination of plasma glucose.

Experiments were designed with a reversal (intervention) paradigm: diabetic rats were untreated for 6 weeks to allow the development of neurovascular dysfunction. They were then treated over the next 2 weeks with a dose of 1 mg/kg/day of a representative compound of the invention, 13-HODE, given as a dietary supplement dispersed in the food with a sunflower oil vehicle (50 ml/2.5 kg food). Groups of nondiabetic control rats and diabetic rats treated with vehicle alone were also studied.

Sciatic Blood Flow

Sciatic nerve endoneurial blood flows in non-diabetic control rats, diabetic rats treated with vehicle alone and diabetic rats treated with 13-HODE were estimated by microelectrode polarography and hydrogen clearance, using the methods described in Day T J, Lagerlund T D, Low P A (1989) Analysis of H₂ clearance curves used to measure blood flow in rat sciatic nerve, J Physiol 414:35-54, and Cameron N E, Cotter M A, Low P A (1991) Nerve blood flow in early experimental diabetes in rats: relation to conduction deficits, Am J Physiol 261:E1-E8. Rats were artificially ventilated. The carotid artery was cannulated to monitor blood pressure, and if necessary rats were given neuromuscular blockade using d-tubocurarine (2 mg kg⁻¹ via the carotid cannula) to reduce mechanical movement artefacts. The level of anaesthesia was monitored by observing any reaction of blood pressure to manipulation, and supplementary thiobutabarbital anaesthetic given as necessary. The target nerve tissue was exposed and the tissue around the incision sutured to a metal ring to form a pool filled with mineral oil at 37° C. During recordings, pool temperature was maintained at 35-37° C. by radiant heat. A glass-insulated platinum microelectrode, polarized at 250 mV with respect to a subcutaneous reference electrode, was inserted into the neural structure. 10% H₂ was added to the inspired gas, the proportions of O₂ and N₂ being adjusted to 20% and 70% respectively. When the H₂ current recorded by the electrode had stabilized, indicating equilibrium with arterial blood, the H₂ supply was shut off and N₂ delivery increased appropriately. H₂ clearance was recorded until a stable baseline was reached, which was defined as no systematic decline in electrode current over 2 min. This procedure was then repeated at another neural site. After the experiment, clearance curves were digitized and mono- or bi-exponential curves fitted to the data by computer using non-linear regression analysis and the general bi-exponential equation:

y=aexp(−bx)+cexp(−dx)+e

where y is the electrode hydrogen current (arbitrary units), x is time (min), a and c are weighting constants for fast (non-nutritive) and slow (nutritive) clearance components respectively, b is the fast component and d is the slow component (ml min⁻¹ ml nerve⁻¹), and e is the baseline electrode current (arbitrary units). Assuming a tissue density of 1, nutritive blood flow was calculated as d×100 (ml min^{'1} 100 g⁻¹). The averages from the two determinations were taken to represent nerve tissue blood flow parameters.

The results of Example 4 are shown as FIG. 7.

It can be seen that sciatic nerve endoneurial blood flow was halved in diabetic rats, and this was completely restored by treatment with 13-HODE.

### Example 5

Groups of non-diabetic control rats, diabetic rats treated with vehicle alone and diabetic rats treated with 13-HODE were obtained as in Example 4.

Before final experiments, nociceptive latencies for withdrawal reflexes to noxious thermal stimulation of the foot were estimated by the Hargreaves plantar test using commercially available equipment (Ugo-Basile, Comerio, Italy). Non-diabetic control rats, diabetic rats treated with vehicle alone and diabetic rats treated with 13-HODE were placed in the thermal testing apparatus, which consisted of a perspex enclosure with a glass base, in which they were free to move. After 30 min acclimatization, a constant power infrared stimulus was focused through the glass base onto the sole of the foot and the latency for reflex foot withdrawal automatically recorded via a photoelectric monitor.

For each session, 4 measurements were obtained, 2 from each foot, the average being taken as the final withdrawal latency.

The results of Example 5 are shown as FIG. 8.

It can be seen from FIG. 8 that there was a decreased latency of response in diabetic rats. This indicates increased sensitivity to potentially noxious heat. This increased sensitivity was completely corrected by treatment with 13-HODE.

### Example 6

Groups of non-diabetic control rats, diabetic rats treated with vehicle alone and diabetic rats treated with 13-HODE were obtained as in Example 4.

Tactile allodynia in non-diabetic control rats, diabetic rats treated with vehicle alone and diabetic rats treated with 13-HODE was monitored using an electronic von Freys hair apparatus. Tests were carried out in a constant temperature room at the same time each day. Allodynia was measured for each foot on one day.

The results of Example 6 are shown as FIG. 9.

It can be seen from FIG. 9 that diabetic rats showed increased tactile allodynia i.e. a reduced threshold for foot withdrawal to tactile stimulation (touch). This means that reflex responses were given to stimuli that were not noxious to nondiabetic rats. Treatment with 13-HODE almost completely reversed this effect.

### Example 7

Groups of non-diabetic control rats, diabetic rats treated with vehicle alone and diabetic rats treated with 13-HODE were obtained as in Example 4.

Before final experiments, nociceptive thresholds for mechanical stimulation were measured by the Randall-Sellito test . Mechanical pressure thresholds in non-diabetic control rats, diabetic rats treated with vehicle alone and diabetic rats treated with 13-HODE were then estimated twice per day for each foot over a 3-day period.

The results of Example 7 are shown as FIG. 10.

It can be seen from FIG. 10 that diabetic rats showed increased sensitivity to mechanical deep pressure. Treatment with 13-HODE resulted in a small but statistically significant improvement in this parameter.

### Example 8

Groups of non-diabetic control rats, diabetic rats treated with vehicle alone and diabetic rats treated with 13-HODE were obtained as in Example 4.

Non-diabetic control rats, diabetic rats treated with vehicle alone and diabetic rats treated with 13-HODE were anaesthetized with thiobutabarbital (50-100 mg kg⁻¹ i.p.). The trachea was cannulated for artificial respiration. The carotid artery was cannulated to monitor systemic blood pressure. The major pelvic ganglion and cavernous nerve in the abdomen were exposed by blunt dissection and bathed in a liquid paraffin pool. Fine bipolar platinum stimulating electrodes were placed around the nerve. The cavernosal space was cannulated using a 23G needle connected to a pressure transducer. Cavernosal pressure responses were recorded in response to 60 s periods of suprathreshold (3-5 mA) nerve stimulation at frequencies in the range 1-32 Hz (stimulus duration 1.5-2 ms). Frequency response curves were then constructed for the area under the pressure development curve, relative to mean systemic pressure, for 75 s from the start of stimulation.

The results of Example 8 are shown as FIG. 11.

FIG. 11 shows that pressure responses depend on the frequency of nerve stimulation during the 60 sec period—the higher the frequency, the bigger the response up to a plateau. There is a marked diabetic deficit at multiple stimulation frequencies, highly statistically significant at 8 Hz and above. This was completely corrected by treatment with 13-HODE. When comparing whole-frequency response curves (i.e. using all the data collected in a single comparison) the 13-HODE treated group curve shows significantly greater pressure response than that of the nondiabetic controls (2-way ANOVA; p<0.01). This is a notable treatment effect.

### Example 9

Experiments were carried out as described in Example 4 above, except that blood flow in the major pelvic ganglion, which houses the cell bodies that give rise to the carvernous nerve fibres supplying the penis, was measured.

The results of Example 9 are shown as FIG. 12.

FIG. 12 clearly shows that blood flow was decreased in diabetic rats, and restored to within the nondiabetic range by treatment with 13-HODE.

### Example 10

An experiment was carried out in accordance with the method of Example 1 to determine the effect of GLA, 13-HODE and 15-HETrE on Motor NCV in diabetic rats.

Dose response curves for Example 10 are shown as FIG. 13.

A measure of the efficacy of the three treatments is given by the ED50 value calculated from the data presented in FIG. 13. The ED50 value for GLA is 164.7 mg/kg. The ED50 value for 13-HODE is 0.057 mg/kg. The ED50 value for 15-HETrE is 0.252 mg/kg.

Thus, 13-HODE is approximately 3000 times more potent than GLA. 15-HETrE is approximately 500 times more potent than GLA.

### Example 11

Levels of 15-HETrE in blood plasma and nerve tissue were determined in populations of rats treated for two weeks with (i) 15-HETrE, (ii) 13-HODE, and (iii) sunflower oil placebo.

The mean 15-HETrE level in population (i) was found to be 1.28 μL (standard deviation 0.83). The mean 15-HETrE level in population (ii) was found to be 0.57 μL (standard deviation 0.33). The mean 15-HETrE level in population (iii) was found to be 0.26 μL (standard deviation 0.30).

These results are shown graphically as FIG. 14.

1-16. (canceled)

17. A method of treating or preventing nerve damage in a mammal, which method comprises administering to said mammal a therapeutically effective amount of a compound which is a polyunsaturated fatty acid (PUFA) derivative of formula (I), 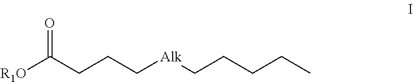
in the form of a racemate, a stereoisomer or a mixture of stereoisomers, or a pharmaceutically acceptable salt, or solvate thereof, wherein
-Alk- is —(CH₂)₄—CH(OR₂)-[trans]CH═CH-[cis]CH═CH—, —(CH₂)₄-[cis]CH═CH-[trans]CH═CH—CH(OR₂)—, —CH(OR₂)-[trans]CH═CH-[cis]CH═CH—CH₂-[cis]CH═CH—(CH₂)₃—, —(CH₂)₃—CH(OR₂)-[trans]CH═CH-[cis]CH═CH—CH₂-[cis]CH═CH—, or —(CH₂)₃-[cis]CH═CH—CH₂-[cis]CH═CH-[trans]CH═CH—CH(OR₂)—;
R₁ is a hydrogen atom; or
R₁ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group; or
R₁ is a group of formula —CH₂—CH(OR₃)—CH₂—(OR₄), wherein R₃ and R₄ are each independently hydrogen atoms or —(C═O)—R₆, wherein R₆ is an aliphatic group having from 3 to 29 carbon atoms; or
R₁ is a group of formula —(CH₂OCH₂)ₘOH, wherein m is an integer of from 1 to 200; or
R₁ is a drug moiety;
each R₂ is the same or different and each independently represents a hydrogen atom; or
a group —(C═O)—R₅, wherein R₅ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group, or R₅ is an aliphatic group having from 3 to 29 carbon atoms, or R₅ is a drug moiety; or
a group of formula —(CH₂OCH₂)ₙOH, wherein n is an integer of from 1 to 200; or
a drug moiety;
and wherein
said alkyl, alkenyl, alkynyl and aliphatic groups are the same or different and are each unsubstituted or substituted with 1, 2 or 3 unsubstituted substituents which are the same or different and are selected from halogen, C₁-C₄ alkoxy, C₂-C₄ alkenyloxy, C₁-C₄ haloalkyl, C₂-C₄ haloalkenyl, C₁-C₄ haloalkoxy, C₂-C₄ haloalkenyloxy, hydroxyl, —SR′, and —NR′R″ groups where R′ and R″ are the same or different and represent hydrogen or unsubstituted C₁-C₂ alkyl;
said aryl, heteroaryl, carbocyclyl and heterocyclyl groups are the same or different and are each unsubstituted or substituted by 1, 2, 3 or 4 unsubstituted substituents which are the same or different and are selected from halogen, cyano, nitro, C₁-C₄ alkyl, C₁-C₄ alkoxy, C₂-C₄ alkenyl, C₂-C₄ alkenyloxy, C₁-C₄ haloalkyl, C₂-C₄ haloalkenyl, C₁-C₄ haloalkoxy, C₂-C₄ haloalkenyloxy, hydroxyl, C₁-C₄ hydroxyalkyl, —SR′ and —NR′R″ groups wherein each R′ and R″ is the same or different and represents hydrogen or unsubstituted C₁-C₄ alkyl.

18. A method according to claim 17, wherein R₁ is hydrogen.

19. A method according to claim 17, wherein R₂ is hydrogen.

20. A method according to claim 17, wherein -Alk- is —(CH₂)₄-[cis]CH═CH-[trans]CH═CH—CH(OR₂)— or —(CH₂)₃-[cis]CH═CH—CH₂-[cis]CH═CH-[trans]CH═CH—CH(OR₂)—.

21. A method according to claim 20, wherein -Alk- is —(CH₂)₃-[cis]-CH═CH—CH₂-[cis]-CH═CH-[trans]-CH═CH—CH(OR₂)—.

22. A method according to claim 17, wherein the PUFA derivative is present as the R enantiomer.

23. A method according to claim 17, wherein the PUFA derivative is present as the S enantiomer.

24. A method according to claim 17, wherein the mammal is a human.

25. A method according to claim 17, wherein the compound is administered orally, parenterally or intravenously.

26. A method according to claim 17, wherein the nerve damage is peripheral neuropathy.

27. A method according to claim 26, wherein the peripheral neuropathy is diabetic neuropathy.

28. A method according to claim 27, wherein the diabetic neuropathy is diabetic neuropathy of the sensory nerves, motor nerves, and/or autonomic nerves.

29. A method of treating or preventing a disease or condition in a mammal which is dizziness, indigestion, bladder infections, foot sores, wastage of thigh muscles, sexual dysfunction, numbness, burning sensations, pain, tingling in the legs and feet, decreased temperature perception, decreased ankle reflex and/or decreased sensitivity to vibrations, arising from diabetic neuropathy, which method comprises administering to said mammal a therapeutically effective amount of a compound which is a polyunsaturated fatty acid (PUFA) derivative of formula (I): 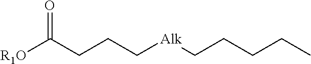
in the form of a racemate, a stereoisomer or a mixture of stereoisomers, or a pharmaceutically acceptable salt, or solvate thereof, wherein
-Alk- is —(CH₂)₄—CH(OR₂)-[trans]CH═CH-[cis]CH═CH—, —(CH₂)₄-[cis]CH═CH-[trans]CH═CH—CH(OR₂)—, —CH(OR₂)-[trans]CH═CH-[cis]CH═CH—CH₂-[cis]CH═CH—(CH₂)₃—, —(CH₂)₃—CH(OR₂)-[trans]CH═CH-[cis]CH═CH—CH₂-[cis]CH═CH—, or —(CH₂)₃-[cis]CH═CH—CH₂-[cis]CH═CH-[trans]CH═CH—CH(OR₂)—;
R₁ is a hydrogen atom; or
R₁ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group; or
R₁ is a group of formula —CH₂—CH(OR₃)—CH₂—(OR₄), wherein R₃ and R₄ are each independently hydrogen atoms or —(C═O)—R₆, wherein R₆ is an aliphatic group having from 3 to 29 carbon atoms; or
R₁ is a group of formula —(CH₂OCH₂)ₘOH, wherein m is an integer of from 1 to 200; or
R₁ is a drug moiety;
each R₂ is the same or different and each independently represents hydrogen; or
a group —(C═O)—R₅, wherein R₅ is a C₁-C₆ alkyl, C₂-C₆ alkenyl, C₂-C₆ alkynyl, C₆-C₁₀ aryl, 5- to 10-membered heteroaryl, C₃-C₇ carbocyclyl or 5- to 10-membered heterocyclyl group, or R₅ is an aliphatic group having from 3 to 29 carbon atoms, or R₅ is a drug moiety; or
a group of formula —(CH₂OCH₂)ₙOH, wherein n is an integer of from 1 to 200; or
a drug moiety;
and wherein
said alkyl, alkenyl, alkynyl and aliphatic groups are the same or different and are each unsubstituted or substituted with 1, 2 or 3 unsubstituted substituents which are the same or different and are selected from halogen, C₁-C₄ alkoxy, C₂-C₄ alkenyloxy, C₁-C₄ haloalkyl, C₂-C₄ haloalkenyl, C₁-C₄ haloalkoxy, C₂-C₄ haloalkenyloxy, hydroxyl, —SR′, and —NR′R″ groups where R′ and R″ are the same or different and represent hydrogen or unsubstituted C₁-C₂ alkyl;
said aryl, heteroaryl, carbocyclyl and heterocyclyl groups are the same or different and are each unsubstituted or substituted by 1, 2, 3 or 4 unsubstituted substituents which are the same or different and are selected from halogen, cyano, nitro, C₁-C₄ alkyl, C₁-C₄ alkoxy, C₂-C₄ alkenyl, C₂-C₄ alkenyloxy, C₁-C₄ haloalkyl, C₂-C₄ haloalkenyl, C₁-C₄ haloalkoxy, C₂-C₄ haloalkenyloxy, hydroxyl, C₁-C₄ hydroxyalkyl, —SR′ and —NR′R″ groups wherein each R′ and R″ is the same or different and represents hydrogen or unsubstituted C₁-C₄ alkyl.

30. A method according to claim 29, wherein the disease or condition is erectile dysfunction.
